SEQUENCE LISTING (39 sequences)

SEQ ID NO: 1 - protein, 6 aa, Artificial Sequence
  cell penetrating peptide
RRRRRR

SEQ ID NO: 2 - protein, 7 aa, Artificial Sequence
  cell penetrating peptide
RRRRRRR

SEQ ID NO: 3 - protein, 8 aa, Artificial Sequence
  cell penetrating peptide
RRRRRRRR

SEQ ID NO: 4 - protein, 9 aa, Artificial Sequence
  cell penetrating peptide
RRRRRRRRR

SEQ ID NO: 5 - protein, 10 aa, Artificial Sequence
  cell penetrating peptide
RRRRRRRRRR

SEQ ID NO: 6 - protein, 11 aa, Artificial Sequence
  cell penetrating peptide
RRRRRRRRRRR

SEQ ID NO: 7 - protein, 12 aa, Artificial Sequence
  cell penetrating peptide
RRRRRRRRRRRR

SEQ ID NO: 8 - protein, 16 aa, Artificial Sequence
  cell penetrating peptide
RRRRRRRRRRRRRRRR

SEQ ID NO: 9 - protein, 6 aa, Artificial Sequence
  cell penetrating peptide
RXXXXX

SEQ ID NO: 10 - protein, 7 aa, Artificial Sequence
  cell penetrating peptide
RXXXXXX

SEQ ID NO: 11 - protein, 8 aa, Artificial Sequence
  cell penetrating peptide
RXXXXXXX

SEQ ID NO: 12 - protein, 9 aa, Artificial Sequence
  cell penetrating peptide
RXXXXXXXX

SEQ ID NO: 13 - protein, 10 aa, Artificial Sequence
  cell penetrating peptide
RXXXXXXXXX

SEQ ID NO: 14 - protein, 8 aa, Artificial Sequence
  cell penetrating peptide
RRRRRRRW

SEQ ID NO: 15 - protein, 13 aa, Artificial Sequence
  cell penetrating peptide
GRRRRRRRRRPPQ

SEQ ID NO: 16 - protein, 23 aa, Artificial Sequence
  cell penetrating peptide
GYGRKKRRGRRRTHRLPRRRRRR

SEQ ID NO: 17 - protein, 11 aa, Artificial Sequence
  cell penetrating peptide
YGRRARRRRRR

SEQ ID NO: 18 - protein, 11 aa, Artificial Sequence
  cell penetrating peptide
YGRRRRRRRRR

SEQ ID NO: 19 - protein, 11 aa, Artificial Sequence
  cell penetrating peptide
YRRRRRRRRRR

SEQ ID NO: 20 - protein, 22 aa, Artificial Sequence
  cell penetrating peptide
AAVALLPAVLLALLAPRRRRRR

SEQ ID NO: 21 - protein, 19 aa, Artificial Sequence
  cell penetrating peptide
XXXXXXXGXGXXXXXXXXX

SEQ ID NO: 22 - protein, 28 aa, Artificial Sequence
  cell penetrating peptide
RRRRRRRRRRRTYADFIASGRTGRRNAI

SEQ ID NO: 23 - protein, 23 aa, Artificial Sequence
  cell penetrating peptide
GYGRKKRRGRRRTHRLPRRRRRR

SEQ ID NO: 24 - protein, 9 aa, Artificial Sequence
  cell penetrating peptide
XRRRRRRRX

SEQ ID NO: 25 - protein, 9 aa, Artificial Sequence
  cell penetrating peptide
XRXRXRXRX

SEQ ID NO: 26 - protein, 13 aa, Artificial Sequence
  cell penetrating peptide
GRRRRRRRRRPPQ

SEQ ID NO: 27 - protein, 17 aa, Artificial Sequence
  cell penetrating peptide
RRRRNRTRRNRRRVRGC

SEQ ID NO: 28 - protein, 19 aa, Artificial Sequence
  cell penetrating peptide
TRQARRNRRRRWRERQRGC

SEQ ID NO: 29 - protein, 28 aa, Artificial Sequence
  cell penetrating peptide
RRRRRRRRRRRTYADFIASGRTGRRNAI

SEQ ID NO: 30 - protein, 36 aa, Artificial Sequence
  cell penetrating peptide
RGSRRAVTRAQRRDGRRRRRSRRESYSVYVYRVLRQ

SEQ ID NO: 31 - protein, 60 aa, Artificial Sequence
  cell penetrating peptide
SKRTRQTYTRYQTLELEKEFHFNRYITRRRRIDIANALSLSERQIKIWFQNRRMKSKKDR

SEQ ID NO: 32 - protein, 16 aa, Artificial Sequence
  cell penetrating peptide
YIVLRRRRKRVNTKRS

SEQ ID NO: 33 - protein, 17 aa, Artificial Sequence
  cell penetrating peptide
DRRRRGSRPSGAERRRR

SEQ ID NO: 34 - protein, 22 aa, Artificial Sequence
  cell penetrating peptide
DRRRRGSRPSGAERRRRRAAAA

SEQ ID NO: 35 - protein, 17 aa, Artificial Sequence
  cell penetrating peptide
TRQARRNRRRRWRERQR

SEQ ID NO: 36 - protein, 4 aa, Artificial Sequence
  cell penetrating peptide
RRRR

SEQ ID NO: 37 - protein, 5 aa, Artificial Sequence
  cell penetrating peptide
RRRRR

SEQ ID NO: 38 - protein, 9 aa, Artificial Sequence
  cell penetrating peptide
CCRRRRRRR

SEQ ID NO: 39 - protein, 9 aa, Artificial Sequence
  cell penetrating peptide
XXXXXXXXX

(19) United States
(12) Patent Application Publication
(10) Pub. No.: US 2017/0114093 A1
(43) Pub. Date: Apr. 27, 2017

(54) Methods of Treatment and Prevention of Disease by Arginine-rich Compositions that Induce Cytoprotection and Neuroprotection

(71) Applicant: Andrew Peter Mallon, Lincoln, RI (US)

(72) Inventor: Andrew Peter Mallon, Lincoln, RI (US)

(21) Appl. No.: 15/168,198

(22) Filed: May 30, 2016

Related U.S. Application Data
(60) Provisional application No. 62/168,365, filed on May 29, 2015.

(51) Int. Cl. C07K 7/06

(52) U.S. Cl. CPC C07K 7/06; A61K 38/00

(57) ABSTRACT

The present invention refers to a composition of arginine-rich poly-peptides, compounds and materials and their use in a method for treatment, amelioration or prevention of cell death associated medical conditions by the provision of a protectant therapy, including cytoprotection and neuroprotection.

### FIELD OF THE INVENTION

This invention was made with US government support under grant number 1R43NS074651-01 awarded by the National Institutes of Health. The government has certain rights in the invention.

The present disclosure generally relates to arginine-containing cell permeant compounds, comprising multiple arginine amino acids that constitute a novel cytoprotective and neuroprotective therapeutic. The invention further relates to pharmaceutical compositions comprising said compounds and methods for the preparation and use of said pharmaceutical corn positions.

### BACKGROUND OF THE INVENTION

US20030091601 described methods of use using the amino acid arginine in topical wound healing.

U.S. Pat. No. 7,557,087 and US20040082659 disclose compositions and methods for treatment of vascular conditions. The invention provides arginine polymers and arginine homopolymers for the treatment and/or prevention of a limited series of indications comprising glaucoma, pulmonary hypertension, asthma, chronic obstructive pulmonary disease (COPD), erectile dysfunction, Raynaud's syndrome, heparin overdose, vulvodynia, and wound healing. This invention also provided arginine polymers and arginine homopolymers for use in organ perfusate and preservation solutions. Importantly, the embodiment and claims described for this invention involve the use of arginine to affect vascular tone. For example, the treatment of glaucoma was stated as by reduction of intraocular pressure, distinct from the cytoprotective and neuroprotective function described in the present invention. Prior art documents describing methods of use for treating diseases are based upon influences on vascular tone, whereas this invention specifically describes a novel and inventive distinct action of cellular cytoprotection and neuroprotection that is independent of vascular tone.

WO2013158739 describes a method of use of arginine peptides with N-terminal cysteine residues and arginine peptides and claims utility by neuroprotection through unsubstantiated references to observations in in vivo vascularized retinal preparations. However, WO2013158739 does not contain an enabling disclosure. Additionally, the present invention specifically excludes those arginine peptides with cysteine residues claimed in WO2013158739. The present invention observed no utility by way of neuroprotection in cortical neurons by the cysteine-arginine peptides described. As described, supra, arginine has a direct and prior art effect upon vasculature as found in the intact eye that produces an obvious neuroprotective effect. WO2013158739 references, but does not disclose, the use of an in vivo eye model to establish an inventive neuroprotective effect. In the WO2013158739 eye model, the vasculature would be intact and there is no evidence that the effect described is inventive, novel or non-obvious.

### SUMMARY OF THE INVENTION

According to one aspect of the present invention, there is provided a method of protecting cells that are susceptible to cell death as a result of physiological or pathological causes in a mammalian subject comprising administering a cell penetrating peptide comprising at least four contiguous arginine residues to said subject to prevent, treat or ameliorate cell death.

According to another aspect of the present invention, there is provided a method of preventing, treating or ameliorating a condition selected from the group consisting pain and/or inflammation (for example, but not limited to, arthritis, retinopathy, SLE, psoriasis, Bullous pemphigoid, shingles or a similar condition), microvascular insufficiency, hypoxia, myocardial infarction, stroke, subarachnoid hemorrhage, atherosclerosis or other acute or chronic neurological ischemic events, mild to severe traumatic brain injury including diffuse axonal injury, hypoxic-ischemic encephalopathy and other forms of craniocerebral trauma, ischemic infarction, embolism and hemorrhage, e.g., hypotensive hemorrhage, neurodegenerative diseases including Alzheimer's disease, Lewy Body dementia, Parkinson's disease (PD), Huntington's disease (HD), multiple sclerosis (MS), amyotrophic lateral sclerosis, Nieman-Pick disease, diabetic neuropathy, neuropathic pain, macular degeneration (wet and dry AMD), retinitis pigmentosa, motor neuron disease, muscular dystrophy, hearing loss, peripheral neuropathies, metabolic disorders of the nervous system including glycogen storage diseases, and other conditions where neurons are damaged or destroyed, abnormal immune activation, such as autoimmune SLE rheumatoid arthritis, Bullous pemphigoid, HIV-associated disorders, AIDS, Type-I diabetes and the like, and conditions characterized by insufficient immune function. Other diseases that may be subject to treatment with compositions of the present invention include psychiatric disorders such as attention deficit hyperactive disorder, depression (in all forms), agoraphobia, bulimia, anorexia, bipolar disorder, anxiety disorder, autism, dementia, dissociative disorder, hypochondriasis, impulse control disorder, kleptomania, mood disorder, multiple personality disorder, chronic fatigue syndrome, insomnia, narcolepsy, schizophrenia, substance abuse, post-traumatic stress disorder, obsessive-compulsive disorder and manic depression, radiation, chemical and biological agent damage, as well as complex disorders such as Gulf War Syndrome or such-like syndromes. Compounds of the present invention can also be used to improve outcomes regarding addiction/addiction recovery. In certain embodiments, compounds of the present invention can also be used to decrease (e.g., inhibit) cell proliferation, including but not limited to cancer.

Diseases, senescence, trauma or ischemic injuries to the brain, eye, ear, or spinal cord often produce permanent damage to neurons and cells. These injuries are serious medical problems with no effective pharmacological treatments. For example, ischemic cerebral stroke, sub-arachnoid hemorrhage or spinal cord injuries manifest themselves as acute loss of neurological capacity, encompassing small focal to global dysfunction, and sometimes leading to death. In vivo, a local decrease in CNS tissue vascular perfusion mediates neuronal death in both hypoxic and traumatic CNS injuries. Local ischemia is often caused by a disruption of the local vasculature, vessel thrombosis, vasospasm, or luminal occlusion by an embolic mass. This ischemia is widely understood to damage susceptible neurons disrupting a variety of cellular homeostatic mechanisms and triggering apoptopic and necrotic cell death signaling events. Treatment comprising administering a cell penetrating peptide comprising at least four contiguous arginine residues to subject in need of such treatment.

According to another aspect of the present invention, there is provided a method of protecting cells that are susceptible to cell death as a result of physiological or pathological causes in a mammalian subject comprising administering a pharmaceutical composition comprising a cell penetrating peptide comprising at least four contiguous arginine residues and a pharmaceutically acceptable carrier to said subject to prevent, treat or ameliorate cell death.

According to still another aspect of the present invention, there is provided a method of preventing, treating or ameliorating a condition selected from the group consisting inflammatory disorders, macular degeneration, hearing loss, diabetic neuropathy, neuropathic pain, AIDS; neurodegenerative diseases such as Alzheimer's disease, Parkinson's disease, and amyotrophic lateral sclerosis; ischemic injury after myocardial infarction, stroke, and reperfusion; and in autoimmune diseases such as hepatitis and graft versus host disease comprising administering a pharmaceutical composition comprising a cell penetrating peptide comprising at least four contiguous arginine residues and a pharmaceutically acceptable carrier to subject in need of such treatment

### DETAILED DESCRIPTION OF THE INVENTION

This invention relates to arginine-containing polypeptides that, in a preferred embodiment of use, provide a protective effect to mammalian cells that are susceptible to cell death as a result of physiological, environmental, traumatic or pathological causes that are hence provided therapy through cytoprotective and neuroprotective pathways of the arginine-containing molecules, as described below.

Peptides, chimeric derivatives, peptidomimetics, as well as compositions containing the same are also provided.

The terms cytoprotective and neuroprotective can be synonymous when describing neurons (neuroprotective). Neuroprotection is a subset of cytoprotection which is defined by a protective effect upon any cell and not just neurons (neuroprotective). Without being bound by any particular theory, nitric oxide (NO) is synthesized, at least partially, from L-arginine by a group of enzymes called nitric oxide synthases (NOS). NOS converts L-arginine, NADPH, and oxygen into L-citrulline, NADH, and NO. NOS occurs endogenously in three isoforms: an endothelial nitric oxide synthase (eNOS), inducible nitric oxide synthase (iNOS), and a neuronal nitric oxide synthase (nNOS). eNOS has been detected in endothelial cells and blood vessels, but also in epithelium of tissues, including bronchial cells and neurons of the brain, especially in the pyramidal cells of the hippocampus. Furthermore, iNOS has been detected not only in macrophages, but also in cells such as hepatocytes, chrondrocytes, endothelial cells, and fibroblasts, in particular under conditions of endothelial damage or as part of a response to injury.

Without being bound by any particular theory, the NOS isoforms can also be categorized as either constitutive or inducible. Constitutive NOS (cNOS) include eNOS and nNOS, while iNOS is inducible. cNOS are usually present in a cell, but remain inactive until intracellular calcium levels increase resulting in enhanced calcium/calmodulin binding and subsequent activation. Unlike cNOS, iNOS is calcium independent and is not normally present in cells. However, iNOS can be induced by lipopolysaccharides and certain cytokines. Cytokine activity affects gene expression/splicing, mRNA stability, and protein synthesis, resulting in iNOS activation and the production of NO. It is also expected that the induced form of NOS produces a much greater amount of NO than cNOS and may even result in toxicity when the L-arginine supply is limited. Induction of iNOS can be inhibited by glucocorticoids and some cytokines (Kroncke K et al., Clinical & Experimental Immunology, 1998, 113:147-56).

In the mammalian CNS, the N-methyl-D-aspartate receptor (NMDAR) is generally understood by those skilled in the art of NMDA research to trigger intracellular signaling pathways governing excitotoxicity apoptosis, necrosis, neuronal plasticity, development, senescence, and disease. The field is recognized as complex with many knowns and unknowns. Studies have been undertaken describing the role of excitotoxic NMDAR signaling by suppressing the expression of the NMDAR scaffolding protein PSD-95 (Aarts M et al., Science, 2002, 298:846-50). Suppressing PSD-95 selectively blocked Ca²⁺-activated NO production by NMDARs, but not by other pathways, without affecting nNOS expression or function. Thus, PSD-95 is required for the efficient coupling of NMDAR activity to NO toxicity and imparts specificity to excitotoxic Ca²⁺ signaling. The dual role of NO eliciting toxicity and protection can be accounted for by ischemic preconditioning (Scorziello A et al., J. Neurochem., 2007, 103:1472-80).

Distinct to the present invention described herein, administration of L-arginine has been shown to increase cerebral blood flow and reduce neurological damage after experimental traumatic brain injury through an action on vascular tone referenced above (Cherian L et al., J. Pharmacol. And Exp. Therapeut., 2003, 304:617-23). In this invention a protective effect that is independent of vasculature and the effect of L-arginine mediated is described. A cultured brain model was employed.

In a certain embodiment, without being bound by any particular theory, the neuroprotective effects of arginine containing peptides, compounds and materials may occur via NO production, as arginine is the immediate precursor of NO in the reaction mediated by the enzyme NOS. However, other mechanisms of action cannot and are not excluded from this invention. NO is synthesized by numerous different tissues and has many physiological functions as well as some pathological effects. The neuroprotective effects of arginine administration could also occur from other effects, including that arginine is essential for the function of certain K_{ATP} channels. Other currently described mechanisms are provided as examples below.

The protective effect of NO can also be related to inhibition of the NMDA receptor. The mechanisms of the effect, however, remain controversial. NO nitrosylates thiol residues of the redox modulatory site lead to the formation of disulfide bonds. Such modification would permanently inhibit Ca²⁺ flux through the channel and decrease NMDA-mediated neurotoxicity. Another hypothesis is that NO, or a derived species perhaps a NO-metal complex, exerts an allosteric action on the NMDA-receptor protein that facilitates the blockade of the receptor by divalent ions.

NO has also been reported to ameliorate the neurotoxicity produced by H₂O₂. Although well known to promote oxidative damage, NO, under certain conditions, can counteract the deleterious effect of reactive oxygen species. NO might divert superoxide anion from other cellular targets. NO reacts with alkoxyl and peroxyl radicals thereby inhibiting radical-chain propagation reactions.

Under certain conditions, NO might act as an OH scavenger, thereby inhibiting radical chain propagation reactions.

NO is also involved in the mechanisms underlying a phenomenon known as ischemic preconditioning (IPC) which has been demonstrated in the brain and heart to provide a protective influence against ischemia and other cell death inducing stimuli that would otherwise result in cell death (Scorziello A at al., J. Neurochem., 2007, 103:1472-80). This has been postulated to be due to a nNOS-Ras-ERK1/2 pathway.

The method of use of arginine-rich peptides, compounds and materials as a therapy that provides cellular protection (cytoprotection) and specifically including neuronal cell protection (neuroprotection) is provided. The field of arginine therapy is recognized as complex with many knowns and unknowns. Arginine-rich peptides, compounds and materials have inherent cell-penetrating abilities through poly-arginine's ability to be uptaken into cells. In a preferred embodiment, arginine-rich peptides, compounds and materials may provide a source of arginine as the peptides are broken down by normal cellular processes to release individual arginine molecules.

Arginine provides a substrate for conversion to citrulline releasing NO which provides the observed therapeutic effect.

This invention of method of use of arginine-rich peptides, compounds and materials provides a substantially drug-like NO based cytoprotective and/or neuroprotective therapy.

As shown in the accompanying drawings, arginine-rich peptides, compounds and materials of the described invention provided neuroprotection to vasculature-free primary cortical rat neurons and suppressed cell death signaling, including JNK (c-Jun N-terminal kinase), a prominent mediator of cell death signaling processes within the cell (Bessero A-C et al., J. Neurochem., 2010, 113:1307-18). JNK signaling is a reliable surrogate marker for cell death processes. This invention does not describe any direct effect upon JNK signaling.

The cytoprotective and neuroprotective effect described herein encompasses broad embodiments where stimuli from out with or within the cell (including a neuronal cell) elicit physiological, pharmacological and pathological stimuli that can lead to the activation of cell death pathways which can lead to cell death. These cell death pathways can include ischemic cell death (oncosis), apoptopic (programmed) and accidental or necrotic (non-programmed) cell death pathways that lead to cell death and/or injury (Majno G and I J, Am. J. Pathol., 1995, 146:3-15). This cell death and injury, that the current invention is protective of, can be a consequence of a wide variety of stimuli including, but not limited to, toxins, heat, gluococorticoids, radiation, nutrient deprivation, viral infection, hypoxia, increased calcium concentrations, pH changes, trauma, cancer, reactive oxygen species, tumor necrosis factor (TNF), and caspase.

The protective effect provided by the current invention can protect cells from cell death and can reverse the effects of cell death in cells that are undergoing cell death but have not yet passed a point in the cell death signaling pathway at which the cell cannot recover.

Preferably, synonymous amino acid residues, which are classified into the same groups and are typically interchangeable by conservative amino acid substitutions, defined in Table 1.

TABLE 1
Preferred Groups of Synonymous amino acid residues.

| Ammo Acid | Synonymous Residue |
|---|---|
| Ser | Ser, Thr, Gly, Asn |
| Arg | Arg, Gln, Lys, Glu, His |
| Leu | Ile, Phe, Tyr, Met Val, Leu |
| Pro | Gly, Ala, (Thr), Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, (Thr), Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, (Thr), Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

In agreement with this invention, peptide derivatives of the invention are also described. As referred to herein, a peptide derivative is a molecule that retains sufficient or all the properties of the primary amino acids of the peptide, however, for example, the N-terminus, C-terminus, and/or one or more of the side chains of the amino acids therein are altered or derivatized chemically. Such peptide derivatives include, for example, but are not limited to, naturally occurring amino acid derivatives, for example, but not limited to, 5-hydroxylysine for lysine, 4-hydroxyproline for proline, ornithine for lysine, homoserine for serine, and suchlike. Additional modifications or derivatives include but are not limited to a label, such as tetramethylrhodamine or fluorescein; or one or more post-translational modifications such as acetylation, amidation, formylation, hydroxylation, methylation, phosphorylation, sulfatation, glycosylation, myristoylation or lipidation. Indeed, certain chemical modifications, in particular N-terminal acetylation and C terminal amidation, have been demonstrated to increase the stability of peptides in human serum.

There are clear advantages for using a mimetic of a given peptide. For example, there are considerable potential time, resource, and cost savings and improved patient compliance associated with peptidomimetics, since they may be manufactured more simply, and/or administered orally, or in depot injections compared with the typical dose routes of parenteral, topical or inhaled administration for peptides. Furthermore, peptidomimetics are potentially less expensive and easier to manufacture than peptides and such derivatives can be specifically modified to improve pharmacokinetics, pharmacodynamics, and safety and tolerability profiles.

In a particular embodiment, the present invention also includes peptidomimetics of the peptides described herein. Peptidomimetics refer to a synthetic chemical compound that has substantially the same structural and/or functional characteristics of the peptides of the invention. The peptidomimetic can be entirely composed of synthetic, non-natural amino acid analogues, or can be a chimeric molecule including one or more natural peptide amino acids and one or more non-natural amino acid analogs. The peptidomimetic can also be composed of any number of natural amino acid conservative substitutions as long as such substitutions do not destroy the activity of the mimetic. Routine testing can be used to determine whether a mimetic has the requisite activity, e.g., that it can provide neuroprotection, cytoprotection, and/or apoptotic functions. The phrase “substantially the same,” when used in reference to a mimetic or peptidomimetic, means that the mimetic or peptidomimetic has one or more activities or functions of the referenced molecule.

Therefore, peptides explained herein have usefulness in the development of such small chemical compounds with similar pharmacological activities and therefore with similar therapeutics. The methods of developing peptidomimetics are conventional. Such as, peptide bonds can be substituted by non-peptide bonds or non-natural amino acids that permit the peptidomimetic to assume a comparable structure, and therefore pharmacological activity, to the parent peptide. Further modifications can also be made by replacing chemical groups of the amino acids with other chemical groups of similar structure. The development of peptidomimetics can be aided by determining the tertiary structure of the original peptide, or peptide binding, either free or bound to a target, e.g., protein, by NMR spectroscopy, crystallography and/or computer-aided molecular modeling. These techniques aid in the development of novel compositions targeted for higher potency and/or greater bioavailability and/or greater stability than the original peptide (Dean PM, BioEssays, 1994, 16:683-7). Once a potential peptidomimetic compound is identified, it may be synthesized and assayed using an assay shown herein or any other appropriate cytoprotective or neuroprotective assays.

It will be obvious to one skilled in the art that a peptidomimetic can be designed from any of the peptides described herein. It will furthermore be apparent that the peptidomimetics of this invention can be further used for the development of even more potent non-peptidic compounds, in addition to their utility as drugs.

Peptidomimetic compositions can contain any combination of non-natural structural components, which are typically from at least three structural groups: residue linkage groups other than the natural amide bond (“peptide bond”) linkages; non-natural residues in place of naturally occurring amino acid residues; residues that induce secondary structural mimicry, i.e., induce or stabilize a secondary structure, e.g., a beta turn, gamma turn, beta sheet, alpha helix conformation, and the like; or other changes that confer resistance to proteolysis. For example, a polypeptide can be characterized as a mimetic when one or more of the residues are joined by chemical means other than an amide bond. Individual peptidomimetic residues can be joined by amide bonds, non-natural and non-amide chemical bonds other chemical bonds or coupling means including, for example, glutaraldehyde, N-hydroxysuccinimide esters, glycoyl, bifunctional maleimides, N,N′-dicyclohexylcarbodiimide (DCC) or N,N′-diisopropyl-carbodiimide (DIC). Linking groups alternative to the amide bond include, for example, glycoyl (HO—CH₂—COOH). ketomethylene (e.g., —C(═O)—CH₂— for —C(═O)—NH—), aminomethylene (CH₂—NH), ethylene, olefin (CH—CH), ether (CH₂—O), thioether (CH₂—S), tetrazole (CN₄—), thiazole, retroamide, thioamide, or ester.

As described, a peptidomimetic can be characterized as comprising one or greater non-natural residues in place of naturally occurring amino acid residue(s). Non-natural residues are known in the art. Particular non-limiting examples of non-natural residues useful as mimetics of natural amino acid residues are mimetics of aromatic amino acids include, for example, but not limited to, D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; D- or L-p-methoxy-biphenylphenylalanine; and D- or L-2-indole(alkyl)alanines, where an alkyl can be substituted or unsubstituted methyl, ethyl, propyl, hexyl, butyl, pentyl, isopropyl, iso-butyl, sec-isotyl, iso-pentyl, or a non-acidic amino acid. Aromatic rings of a non-natural amino acid that can be used in place a natural aromatic ring include, for example, but not limited to, thiazolyl, thiophenyl, pyrazolyl, benzimidazolyl, naphthyl, furanyl, pyrrolyl, and pyridyl aromatic rings.

Cyclic peptides or cyclized residue side chains also decrease susceptibility of a peptide to proteolysis by exopeptidases or endopeptidases. Thus, certain embodiments embrace a peptidomimetic of the peptides disclosed and described herein, whereby one or more amino acid residue side chains are cyclized according to conventional methods. In addition, back bone cyclization of the entire peptide is a proffered embodiment, in some cases extension of the sequence is employed to conserve pharmacological efficacy

As described, mimetics of acidic amino acids can be generated by substitution with non-carboxylate amino acids while maintaining a negative charge; (phosphono) alanine; and sulfated threonine. Carboxyl side groups (e.g., aspartyl or glutamyl) can also be selectively modified by reaction with carbodiimides (R′—N—C—N—R′) including, for example, but not limited to, 1-cyclohexyl-3(2-morpholinyl-(4-ethyl) carbodiimide or 1-ethyl-3(4-azonia-4,4-dimetholpentyl) carbodiimide. Aspartyl or glutamyl groups can also be converted to asparaginyl and glutaminyl groups by reaction with ammonium ions.

In certain embodiments, lysine mimetics can be generated (and amino terminal residues can be altered) by reacting lysinyl with succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate.

Methionine mimetics can be generated by reaction with methionine sulfoxide. Proline mimetics of include, for example, but not limited to, pipecolic acid, thiazolidine carboxylic acid, dehydroproline, 3- or 4-methylproline, and 3,3,-dimethylproline.

One or more residues can also be replaced by an amino acid (or peptidomimetic residue) of the opposite chirality. Thus, any amino acid naturally occurring in the L-configuration (which can also be referred to as R or S, depending upon the structure of the chemical entity) can be replaced with the same amino acid or a mimetic, but of the opposite chirality, referred to as the D-amino acid, and that can additionally be referred to as the R- or S-form.

As will be obvious to one skilled in the art, the peptidomimetics of the present invention can also include one or more of the modifications described herein for derivatized peptides, e.g., a label, one or more post-translational modifications, or cell-penetrating sequence. While a peptide of this invention can be derivatized with by one of the above indicated modifications, it is understood that a peptide of this invention may contain more than one of the above described modifications within the same peptide.

In an embodiment, a cell penetrating peptide includes 4 to 100 contiguous arginine residues. In an embodiment, a cell penetrating peptide comprises 4 to 50 contiguous arginine residues. In an embodiment, a cell penetrating peptide comprises 4 to 30 contiguous arginine residues. In an embodiment, a cell penetrating peptide comprises 5 to 20 contiguous arginine residues. In an embodiment, a cell penetrating peptide comprises 6 to 16 contiguous arginine residues. In an embodiment, a cell penetrating peptide comprises 6 to 12 contiguous arginine residues. In an embodiment, a cell penetrating peptide comprises 6 to 10 contiguous arginine residues. In an embodiment, a cell penetrating peptide comprises 6 contiguous arginine residues. In an embodiment, a cell penetrating peptide comprises 7 contiguous arginine residues. In an embodiment, a cell penetrating peptide comprises 8 contiguous arginine residues. In an embodiment, a cell penetrating peptide comprises 9 contiguous arginine residues. In an embodiment, a cell penetrating peptide comprises 10 contiguous arginine residues.

The contiguous arginine residues can be at the C-terminus, N-terminus or in the center of the polypeptide (e.g., surrounded by non-arginine amino acid residues). Non-arginine residues are preferably amino acids, amino acid derivatives, or amino acid mimetics that do not significantly reduce the solubility or rate of membrane transport of the polymer into cells, including, for example but not limited to, glycine, alanine, cysteine, valine, leucine, isoleucine, methionine, serine, threonine, α-amino-β-guanidinopropionic acid, α-amino-γ-guanidinobutyric acid, and α-amino-ε-guanidinocaproic acid. In preferred embodiments, the arginine polymer does not include lysine and histidine monomers.

In certain embodiments, an arginine polymer can be attached to one or more backbones. A backbone is any assembly that allows for the connection of one or more NO enhancers, arginine polymers, arginine copolymers, and/or arginine homopolymers. The NO enhancers, arginine polymers, copolymer and/or homopolymers can be attached to the backbone covalently or non-covalently, directly or by a linker arm. The backbone can be composed of monomer units that are covalently and/or non-covalently linked. Examples of covalent backbones include, but not limited to, oligosaccharides, peptides, lipids and other cross-linked monomers. Examples of non-covalent backbones include, but not limited to, liposomes, nano-particles, micelles, colloids, protein aggregates, modified cells, and modified viral particles. The backbone can form any structure, including but not limited to, linear, branched, hyperbranched, dendrimer, block, star, graft, derivatized, liposomes, michelles, and colloids, or mixtures of one or more these structures. In preferred embodiments, one or more arginine polymers, copolymers, or homopolymers are attached to a backbone (e.g., oligosaccharide) by a cleavable linker such as an ester linkage. In other preferred embodiments, the backbone may be a liposome, polymer, nanoparticle, material or a micelle that presents on its surface one or more arginine polymers, copolymers, or homopolymers.

In an embodiment, at least 50% of the amino acid residues of the cell penetrating peptide are arginine residues. In another embodiment, at least 60% of the amino acid residues of the cell penetrating peptide are arginine residues. In yet another embodiment, at least 70% of the amino acid residues of the cell penetrating peptide are arginine residues. In a further embodiment, at least 80% of the amino acid residues of the cell penetrating peptide are arginine residues. In yet a further embodiment, at least 90% of the amino acid residues of the cell penetrating peptide are arginine residues. In but another embodiment, 100% of the amino acid residues of the cell penetrating peptide are arginine residues.

An arginine polymer can be comprised of L-arginines, D-arginines, or a combination of both L and D-arginines. The term “poly-L-arginine” refers to a sequence composed of all L-arginines. The term “poly-D-arginine” refers to a sequence composed of all D-arginines. The term “poly-L-arginine” may be abbreviated by an upper case “R” followed by the number of L-arginine in the peptide (e.g., R9 represents a 9-mer of contiguous L-arginine residues). The term “poly-D-arginine” may be abbreviated by the lower case “r” followed by the number of D-arginines in the peptide (e.g., r9 represents a 9-mer of contiguous D-arginine residues). “Ac” indicates a sequence having an acetylated N-terminal residue (e.g., AcR9), while “b” indicates a sequence having a biotinylated N-terminal residue (e.g., bR9).

In an embodiment, an arginine polymer or copolymer comprises at least 50% L-arginine residues. In another embodiment, an arginine polymer or copolymer comprises at least 60% L-arginine residues. In yet another embodiment, an arginine polymer or copolymer comprises at least 70% L-arginine residues. In a further embodiment, an arginine polymer or copolymer peptide comprises at least 80% L-arginine residues. In yet a further embodiment, an arginine polymer or copolymer comprises at least 90% of L-arginine residues. In but another embodiment, an arginine polymer or copolymer comprises all L-arginine residues.

When a polymer comprises only arginine residues, it can be referred to herein as an “arginine homopolymer.” An arginine homopolymer can contain a random mixture of L-arginine and D-arginine residues, or a specified combination of L- and D-arginine residues. In an embodiment, at least 50% of all arginine residues in an arginine homopolymer are L-arginine. In another embodiment, at least 60% of all arginine residues in an arginine homopolymer are L-arginine. In yet another embodiment, at least 70% of all arginine residues in an arginine homopolymer are L-arginine. In a further embodiment, at least 80% of all arginine residues in an arginine homopolymer are L-arginine. In yet a further embodiment, at least 90% of all arginine residues in an arginine homopolymer are L-arginine. In but another embodiment, all arginine residues in an arginine homopolymer are L-arginine.

In an embodiment said cell penetrating peptide is selected from the group consisting of:

| Com- | |
|---|---|
| pound | |
| No: | SEQUENCE |
| 1 | RRRRRR |
| | (SEQ ID NO: 1) |
| 2 | RRRRRRR |
| | (SEQ ID NO: 2) |
| 3 | RRRRRRRR |
| | (SEQ ID NO: 3) |
| 4 | RRRRRRRRR |
| | (SEQ ID NO: 4) |
| 5 | RRRRRRRRRR |
| | (SEQ ID NO: 5) |
| 6 | RRRRRRRRRRR |
| | (SEQ ID NO: 6) |
| 7 | RRRRRRRRRRRR |
| | (SEQ ID NO: 7) |
| 8 | RRRRRRRRRRRRRRRR |
| | (SEQ ID NO: 8) |
| 9 | Rrrrrr |
| | (SEQ ID NO: 9) |
| 10 | Rrrrrrr |
| | (SEQ ID NO: 10) |
| 11 | Rrrrrrrr |
| | (SEQ ID NO: 11) |
| 12 | Rrrrrrrrr |
| | (SEQ ID NO: 12) |
| 13 | Rrrrrrrrrk |
| | (SEQ ID NO: 13) |
| 14 | RRRRRRRW |
| | (SEQ ID NO: 14) |
| 15 | GRRRRRRRRRPPQ |
| | (SEQ ID NO: 15) |
| 16 | GYGRKKRRGRRRTHRLPRRRRRR |
| | (SEQ ID NO: 16) |
| 17 | YGRRARRRRRR |
| | (SEQ ID NO: 17) |
| 18 | YGRRRRRRRRR |
| | (SEQ ID NO: 18) |
| 19 | YRRRRRRRRRR |
| | (SEQ ID NO: 19) |
| 20 | AAVALLPAVLLALLAPRRRRRR |
| | (SEQ ID NO: 20) |
| 21 | kkwkmrrGaGrrrrrrrrr |
| | (SEQ ID NO: 21) |
| 22 | RRRRRRRRRRRTYADFIASGRTGRRNAI |
| | (SEQ ID NO: 22) |
| 23 | GYGRKKRRGRRRTHRLPRRRRRR |
| | (SEQ ID NO: 23) |
| 24 | rRRRRRRRr |
| | (SEQ ID NO: 24) |
| 25 | rRrRrRrRr |
| | (SEQ ID NO: 25) |
| 26 | GRRRRRRRRRPPQ |
| | (SEQ ID NO: 26) |
| 27 | RRRRNRTRRNRRRVRGC |
| | (SEQ ID NO: 27) |
| 28 | TRQARRNRRRRWRERQRGC |
| | (SEQ ID NO: 28) |
| 29 | RRRRRRRRRRRTYADFIASGRTGRRNAI |
| | (SEQ ID NO: 29) |
| 30 | RGSRRAVTRAQRRDGRRRRRSRRESYSV |
| | YVYRVLRQ |
| | (SEQ ID NO: 30) |
| 31 | SKRTRQTYTRYQTLELEKEFHFNRYITR |
| | RRRIDIANALSLSERQIKIWFQNRRMKS |
| | KKDR |
| | (SEQ ID NO: 31) |
| 32 | YIVLRRRRKRVNTKRS |
| | (SEQ ID NO: 32) |
| 33 | DRRRRGSRPSGAERRRR |
| | (SEQ ID NO: 33) |
| 34 | DRRRRGSRPSGAERRRRRAAAA |
| | (SEQ ID NO: 34) |
| 35 | TRQARRNRRRRWRERQR |
| | (SEQ ID NO: 35) |

In an embodiment the cell penetrating peptide is selected from the group consisting of

| Com- | |
|---|---|
| pound | |
| No. | SEQUENCE |
| 36 | RRRR |
| | (SEQ ID NO: 36) |
| 37 | RRRRR |
| | (SEQ ID NO: 37) |
| 38 | RRRRRR |
| | (SEQ ID NO: 1) |
| 39 | RRRRRRR |
| | (SEQ ID NO: 2) |
| 40 | RRRRRRRR |
| | (SEQ ID NO: 3) |
| 41 | RRRRRRRRR |
| | (SEQ ID NO: 4) |
| 42 | RRRRRRRRRRR |
| | (SEQ ID NO: 6) |
| 43 | RRRRRRRRRRRR |
| | (SEQ ID NO: 7) |
| 44 | RRRRRRRRRRRRRRRR |
| | (SEQ ID NO: 8) |

In an embodiment the cell penetrating peptide comprises the amino acid sequence-RRRRRRRRR (SEQ ID: 4).

As is well understood by the person skilled in the art, the one letter code for each amino acid is as follows:

| single-letter | | |
|---|---|---|
| code | abbreviation | full name |
| A | Ala | Alanine |
| R | Arg | Arginine |
| N | Asn | Asparagine |
| D | Asp | Aspartic acid |
| C | Cys | Cysteine |
| Q | Gln | Glutamine |
| E | Glu | Glutamic acid |
| G | Gly | Glycine |
| H | His | Histidine |
| I | Ile | Isoleucine |
| L | Leu | Leucine |
| K | Lys | Lysine |
| M | Met | Methionine |
| F | Phe | Phenylalanine |
| P | Pro | Proline |
| S | Ser | Serine |
| T | Thr | Threonine |
| W | Trp | Tryptophan |
| Y | Tyr | Tyrosine |
| V | Val | Valine |

As is well understood by the person skilled in the art, capital letters denote naturally-occurring L-amino acids whereas lower case letters denote unnatural D-amino acids.

Embodiments with partial or total substitution of D-amino acids to generate the chiral D-form, known to impart stability on peptide drugs (Milton R et al., Science, 1992, 256:1445-8). In addition, D-peptides induce less of an immunogenic reaction, although very small peptides without cysteine such as this are less likely to aggregate and thus also less likely to induce immunogenic reactions (Adessi C and Soto C, Curr. Medicin. Chem., 2002, 9:963-78). Selective enhancement of peptide stability is advantageous to decrease proteolytic enzyme digestion exopeptidases and endopeptidases (Adessi C and Soto C, Curr. Medicin. Chem., 2002, 9:963-78).

D-Retro-Inverso conformations describes analogues with D-amino acids in the reversed sequence. This works best on small peptides like Compound 4, listed above, that do not rely upon a secondary structure for target binding. D-retro-inverso versions of cell permeable peptides (CPPs) have enhanced cell penetration and the added benefit of being protease resistant. Reduced proteolysis also increases intracellular peptide concentration.

A “cell penetrating peptide” as used herein refers to a peptide which includes a cell penetrating peptide sequence of amino acids rich in basic amino acids (arginine and lysine) and have positive net charge or neutral pH that confers cell permeability. These peptides show no cell type specificity and are able to penetrate the plasma membrane in a receptor independent manner without causing significant membrane damage. Sequences according to invention are described, for example, in U.S. Pat. No. 6,593,292 and US-2003-0022831. In these publications, the sequences are disclosed were previously described as having utility as cell penetrating peptides. A collection of cell penetrating peptides (CPPs) is contained at CPPsite (http://crdd.osdd.net/raghava/cppsite/Keyword.php), a database of experimentally validated Cell Penetrating Peptides. CPPs are widely used to promote intracellular uptake of conjugated cargos (nucleic acids, peptide nucleic acids, proteins, drugs, liposomes etc.) and thus play role to overcome the problem of poor delivery and low bioavailability of therapeutic molecules. CPP conjugated drugs when delivered in vivo have shown promising results with high efficacy. CPPsite database's current version contains comprehensive information of eight hundred forty-three (843) CPPs.

Standard Fmoc-based protocols can be used to synthesize CPP peptides (Chan WC and White PD, Fmoc Solid Phase Peptide Synthesis, A Practical Approach, 2000). Peptides are synthesized by using Fmoc/tBu solid phase chemistry. Fmoc-iso-Wang resin is the starting amino acid and the peptide chain is assembled on the resin using coupling and deprotection cycles with HBTU/DIPEA and 20% piperidine in DMF, respectively. The deprotection of the side chain and final cleavage from the solid support using cocktail (water/triisopropylsilane/TFA (2.5:2.5:95, v/v/v, 2 h), affords the crude peptide, which can be purified by HPLC (95%) and lyophilized to give pure peptide. The chemical structure of the peptide can be confirmed by using a high-resolution time-of-flight electrospray mass spectrometer.

Pharmaceutical formulations for use with the invention described herein can be formulated by standard techniques using none, one or more physiologically acceptable carriers or excipients.

The inventions and their physiologically acceptable salts and solvates can be formulated for administration by any suitable route, including but not limited to, by inhalation, topically (e.g. lungs, eye, ear, skin, bowel mucosa), buccal, sublingually, intranasally, intra-vitreally, trans-retinally, trans-sclerally, orally, parenterally (e.g., intravenously, intraperitoneally, intramuscularly, subcutaneously, intravesically or intrathecally), or mucosally (including intranasally, orally, intravaginal, and rectally).

In regards to oral, buccal, or sublingual administration, solid pharmaceutical formulations of compositions of the invention can take the form of, for example, but not limited to, lozenges, tablets, powders or capsules prepared as fast-melt, quick-dissolve, or other targeted process, by conventional means using pharmaceutically acceptable excipients, including binding agents, for example, but not limited to, pre-gelatinized corn starch, polyvinylpyrrolidone, or hydroxypropyl methylcellulose; fillers, for example, but not limited to, lactose, microcrystalline cellulose, or calcium hydrogen phosphate; lubricants, for example, but not limited to, magnesium stearate, talc, or silica; disintegrants, for example, but not limited to, potato starch or sodium starch glycolate; or wetting agents, for example, but not limited to, sodium lauryl sulfate. Tablets can be coated and enveloped by methods well known in the art.

Liquid formulations for oral administration can take the form of, for example, but not limited to, solutions, syrups, gels or colloidal suspensions, or they can be presented as a dry product for constitution with water or other suitable vehicle before use. Such liquid preparations can be prepared by traditional processes with pharmaceutically acceptable additives, for example, but not limited to, suspending agents, for example, but not limited to, sorbitol syrup, cellulose derivatives, or hydrogenated edible fats; emulsifying agents, for example, but not limited to, lecithin or acacia; non-aqueous vehicles, for example, but not limited to, almond oil, oily esters, ethyl alcohol, or fractionated vegetable oils; and preservatives, for example, but not limited to, methyl or propyl-p-hydroxybenzoates or sorbic acid. The formulations can also contain buffer salts, flavoring, coloring, and/or sweetening agents as appropriate. If preferred, preparations for oral administration can be suitably formulated to give controlled, sustained and targeted release of the active compound.

For administration by inhalation, the compounds may be conveniently delivered in the form of an aerosol spray presentation from pressurized packs or a nebulizer, with the use of a suitable propellant, for example, but not limited to, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, but not limited to, gelatin for use in an inhaler or insufflator can be formulated containing a powder mix of the compound and a suitable powder base, for example, but not limited to, lactose or starch.

In a preferred embodiment the invention will be formulated as a dry powder inhaler, that has the potential to provide systemic absorption, and in another embodiment both topical pulmonary and systemic doses, including but not limited to the brain.

The inventions can be formulated for parenteral administration by injection, for example, by bolus injection or infusion for systemic or local instillation. Formulations for injection can be presented in unit dosage form, for example, in ampoules or in single or multi-dose containers, with or without an added preservative. The compositions can take such forms as suspensions, solutions, or emulsions in oily or aqueous vehicles, and can contain formulatory agents, for example, suspending, stabilizing, and/or dispersing agents. In a preferred embodiment, the active ingredient can be in powder form for constitution with a suitable vehicle, for example, but not limited to, sterile pyrogen-free water, dextrose 5%, or 0.9% saline for injection, before use.

The compositions of the invention may also be formulated in rectal or vaginal or intestinal compositions such as liquids or solutions for direct instillation by injection or surface dosing, or as suppositories or retention enemas, for example for the latter, but not limited to, containing conventional suppository bases such as cocoa butter or other glycerides or other commonly used suppository or enema formulations.

The compositions of the invention may also be formulated for transdermal administration. For transdermal administration, the active compounds are formulated into ointments, salves, gels, or creams as generally known in the art with or without penetration enhancers. Pharmaceutical compositions adapted for transdermal administration can be provided as discrete patches intended to remain in intimate contact with the epidermis for a prolonged period of time. These compositions may optionally include penetration enhancers for increasing or enabling improved penetration of the corneum stratum. If the compositions of the invention are to be administered topically, the compositions can be formulated in the form of, for example, but not limited to, an ointment, cream, transdermal patch, lotion, gel, spray, aerosol, solution, emulsion, or other form well-known to one of skill in the art. For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (for example, but not limited to, preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as Freon®), or in a squeeze bottle. Moisturizers or humectants can also be added to pharmaceutical compositions and dosage forms if desired. Examples of such additional ingredients are well-known in the art. Compositions may also be included in a device for transdermal delivery such as a skin patch or a more complex device.

The compositions also may be formulated as a depot preparation for sustained or delayed release specific for the disease indication being targeted. Such delayed-acting or long-acting formulations can be administered by implantation (for example, subcutaneously, intradermally, or intramuscularly) or by subcutaneously, intradermally, or intramuscular injection. Thus, for example, the compounds can be formulated with suitable polymeric or hydrophobic materials (for example as an emulsion or suspension in an acceptable oil or oil and water-soluble mixture) or ion exchange resins, or as sparingly soluble derivatives, for example, as a sparingly soluble salt. In addition, they may be formulated in long-acting implanted formulations that allow for stable formulations to slowly be exposed and released into the systemic circulation or topically.

The compositions may also be in the form of controlled release or sustained release compositions as known in the art, for instance, in matrices of biodegradable or non-biodegradable injectable polymeric microspheres or microcapsules, polymeric nanospheres, nanosuspensions, or nanocapsules in liposomes, in emulsions, and the like.

The compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

Depending on their chemical and physical nature, compounds of the invention may be included in the compositions and administered to the patient per se, or in another form such as a salt, solvate, complex, chelate or other derivative as appropriate or as needed for good formulation or administration of the substance. Likewise, a prodrug of the substance may be included in the compositions, that is, a substance that releases the active substance either on preparation of the composition or on administration of the composition to the patient or subject.

### Indications for Use

In preferred embodiments, the subject compounds may be administered to a subject suffering from pain and/or inflammation (for example, but not limited to, arthritis, retinopathy, SLE, psoriasis, Bullous pemphigoid, shingles or a similar condition), a subject at risk of, or having undergone, microvascular insufficiency, hypoxia, myocardial infarction, stroke, Sub Arachnoid hemorrhage, atherosclerosis or another acute or chronic neurological ischemic events patients with mild to severe traumatic brain injury, including diffuse axonal injury, hypoxic-ischemic encephalopathy and other forms of craniocerebral trauma, patients suffering from ischemic infarction, embolism and hemorrhage, e.g., hypotensive hemorrhage, subjects with neurodegenerative diseases including Alzheimer's disease, Lewy Body dementia, Parkinson's disease (PD), Huntington's disease (HD), multiple sclerosis (MS), amyotrophic lateral sclerosis, Nieman-Pick disease, diabetic neuropathy, neuropathic pain, macular degeneration (wet and dry AMD), retinitis pigmentosa, motor neuron disease, muscular dystrophy, hearing loss, peripheral neuropathies, metabolic disorders of the nervous system including glycogen storage diseases, and other conditions where neurons are damaged or destroyed, patients with abnormal immune activation, such as autoimmune SLE rheumatoid arthritis, Bullous pemphigoid, HIV-associated disorders, AIDS, Type-I diabetes, and the like; while others may include those characterized by insufficient immune function. Other diseases that may be subject to treatment with compositions of the present invention include psychiatric disorders such as attention deficit hyperactive disorder, depression (in all forms), agoraphobia, bulimia, anorexia, bipolar disorder, anxiety disorder, autism, dementia, dissociative disorder, hypochondriasis, impulse control disorder, kleptomania, mood disorder, multiple personality disorder, chronic fatigue syndrome, insomnia, narcolepsy, schizophrenia, substance abuse, post-traumatic stress disorder, obsessive-compulsive disorder, and manic depression, radiation, chemical and biological agent damage, as well as complex disorders such as Gulf War Syndrome, or such-like syndromes. Compounds of the present invention can also be used to improve outcomes regarding addiction/addiction recovery. In certain embodiments, compounds of the present invention can also be used to decrease (e.g., inhibit) cell proliferation, including but not limited to cancer.

Diseases, senescence, trauma or ischemic injuries to the brain, eye, ear, or spinal cord often produce permanent damage to neurons and cells. These injuries are serious medical problems with no effective pharmacological treatments. For example, ischemic cerebral stroke, sub-arachnoid hemorrhage or spinal cord injuries manifest themselves as acute loss of neurological capacity, encompassing small focal to global dysfunction, and sometimes leading to death. In vivo, a local decrease in CNS tissue vascular perfusion mediates neuronal death in both hypoxic and traumatic CNS injuries. Local ischemia is often caused by a disruption of the local vasculature, vessel thrombosis, vasospasm, or luminal occlusion by an embolic mass. This ischemia is widely understood to damage susceptible neurons disrupting a variety of cellular homeostatic mechanisms and triggering apoptopic and necrotic cell death signaling events.

In certain preferred embodiments, the present invention describes a method of treating or preventing a symptom associated with stroke comprising administering an effective amount of a pharmaceutical composition to a subject in need thereof, wherein the pharmaceutical composition comprises any compound of the present invention.

Importantly in this method described herein, the invention provides cytoprotective and neuroprotective effects that prevent, treat or ameliorate cell death. It does not describe an effect that is dependent upon changes in vascular tone as described in prior art (U.S. Pat. No. 7,557,087).

Objectives, features and advantages of the embodiments shall become apparent as the description thereof proceeds when considered in connection with the accompanying illustrative drawings.

### BRIEF DESCRIPTION OF THE DRAWINGS

In the drawings that illustrate several exemplary modes for carrying out the present invention:

FIGS. 1 and 2 are histograms showing the cell viability of primary rat cortical neurons that had been exposed to ninety (90) minutes oxygen glucose deprivation. Pre-incubation of an arginine-rich polypeptide [RRRRRRRRR (SEQ ID: 4)] 2 μM (p<0.05, n=7) or 10 μM (p<0.01, n=7) resulted in significant protection of cortical rat neurons from the impact of OGD. CCRRRRRRR (SEQ ID NO: 38) or ccrrrrrrr (SEQ ID NO: 39), 2 μM or 10 μM provided no significant neuroprotection. The level of protection provided by Compound 4 (SEQ ID NO: 4) was comparable to that provided by CP 101 606 10 μM (AT014) or NVPAAM077 100 nM (AT015), NMDA NR2B and NR2A subunit selective antagonists, respectively.

FIG. 3 shows two histograms depicting Western blot measurements of p54 and p48 JNK signaling. JNK signaling is widely considered to be an indicator of cell death signaling. These measurements were taken before (baseline) and after OGD treatments described in FIG. 1., Compound 4 2 μM significantly suppressed JNK cell death signaling (p<0.05, n=3) to a level comparable to both baseline and that provided by NMDA antagonists known to be effective neuroprotectants (AT014: CP 101 606, 10 μM, AT015: NVP-AAM077, 100 nM).

### DETAILED DESCRIPTION OF A PREFERRED EMBODIMENT

Arginine-containing peptides provide a therapeutic paradigm to provide neuroprotection and cytoprotection. Using standard Fmoc-based protocols, we designed and synthesized a specific arginine rich peptide as an exemplary embodiment of this invention RRRRRRRRR (SEQ ID NO: 4) (arginine being represented by the one-letter symbol ‘R’ and three letter abbreviation Arg) identified herein as Compound 4 (SEQ ID NO: 4).

Example 1 Standard Fmoc-based protocols can be used to synthesize CPP peptides (Chan WC and White PD, Fmoc Solid Phase Peptide Synthesis, A Practical Approach, 2000). Peptides are synthesized by using Fmoc/tBu solid phase chemistry. Fmoc-iso-Wang resin is the starting amino acid and the peptide chain is assembled on the resin using coupling and deprotection cycles with HBTU/DIPEA and 20% piperidine in DMF, respectively. The deprotection of the side chain and final cleavage from the solid support using cocktail (water/triisopropylsilane/TFA (2.5:2.5:95, v/v/v, 2 h), affords the crude peptide, which can be purified by HPLC (95%) and lyophilized to give pure peptide. The chemical structure of the peptide can be confirmed by using a high-resolution time-of-flight electrospray mass spectrometer.

### Example 2

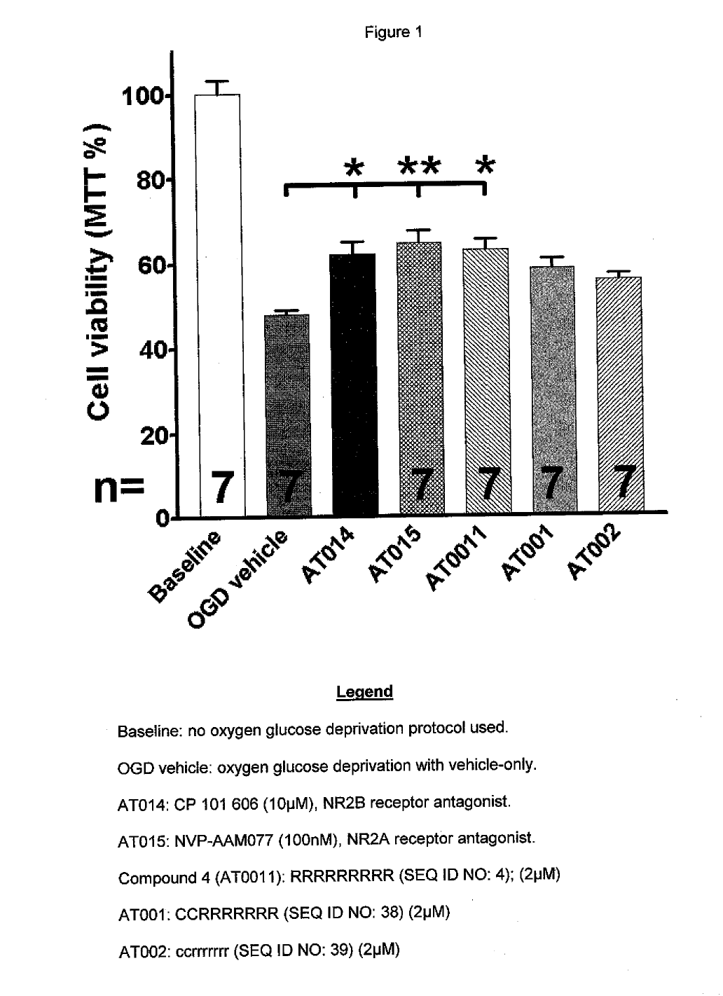

Dissociated primary neuronal cell cultures were prepared from rat brains at 37° C. in a humidified atmosphere containing 5% CO₂ and 95% O₂ and grown to confluence in glucose-containing media. Indicated compounds were applied thirty (30) minutes before cultures were placed in an environment of oxygen glucose deprivation (OGD). OGD was achieved using glucose-free media and a humidified atmosphere containing 5% CO₂ and 95% N₂. OGD was continued for ninety (90) minutes and terminated by return to baseline conditions with glucose and O₂ restored. Reduction of 3-(4,5-dimethyl-2-thiazolyl)-2,5-diphenyltetrazolium bromide (MTT) by cellular dehydrogenases measured mitochondrial activity and cell health. Statistical significance of the difference between populations of neurons under different experimental conditions, as shown in FIG. 1, were calculated using ANOVA and Newman-Keuls Multiple Comparison Test (*; p<0.05, **p<0.01, ***p<0.001). This model is a dissociated cell preparation, therefore vasculature is absent, therefore the activity of Compound 4 (SEQ ID NO: 4), an inventive Arginine rich polypeptide, is shown to be independent of changes to vascular tone and indicates protection from a novel mechanism of action. Control peptides CCRRRRRRR (SEQ ID NO: 38) or ccrrrrrrr (SEQ ID NO: 39) were tested at 2 μM and 10 μM, but showed no significant neuroprotective effect.

### Example 3

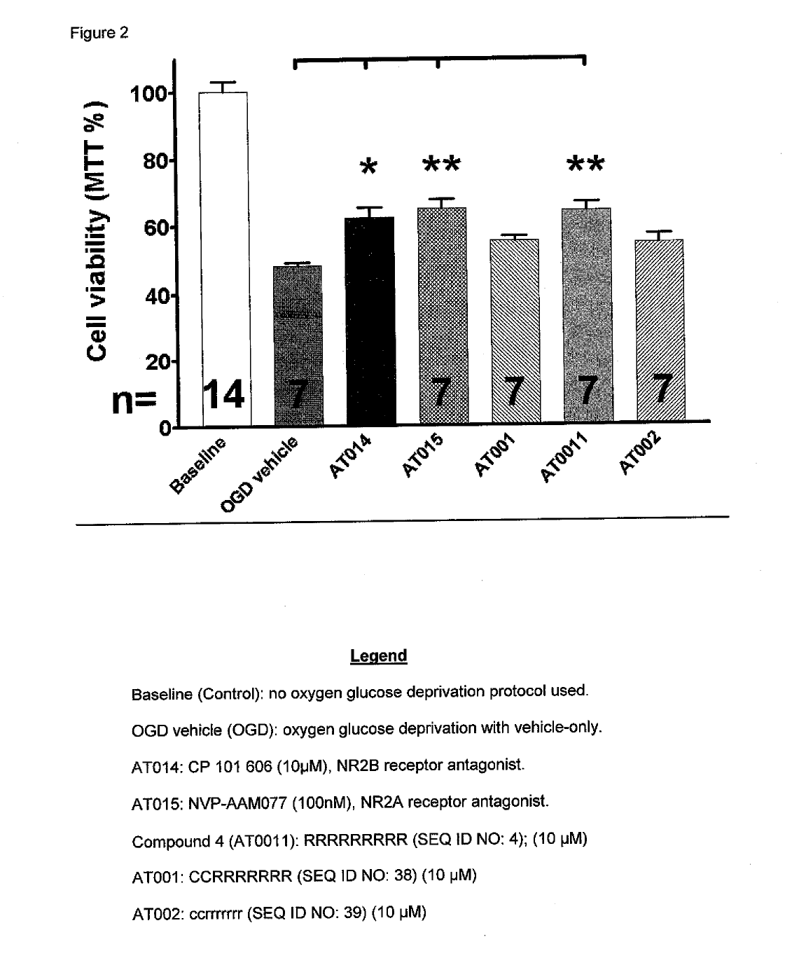

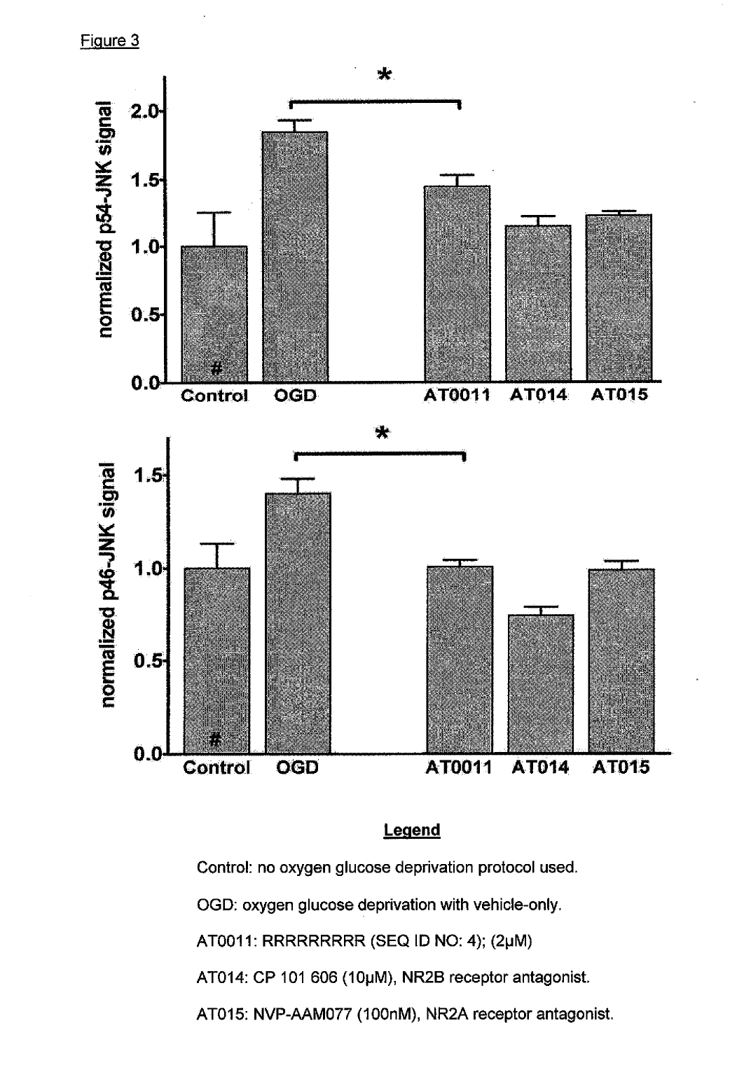

Dissociated primary neuronal cell cultures treated in the same manner as indicated in Example 2 were collected and their cells lysed. Cellular proteins were precipitated and denatured for immunohistochemical analysis by Western Blot. Phospho-SAPK/JNK (Thr202/Tyr204) antibody detects endogenous levels of p46 and p54 SAPK/JNK dually phosphorylated at threonine 183 and tyrosine 185. Levels of The stress-activated protein kinase/Jun-amino-terminal kinase SAPK/JNK are widely considered to be molecular sequelae of the activation of cell death pathways that lead to apoptosis. As shown in FIG. 2, the induction of OGD caused levels of pSAPK/JNK to be increased compared with baseline control. In addition, OGD combined with treatment by the indicated compounds that prevented cell death, concomitantly prevented the activation of the SAPK/JNK cell death pathway.

While there is shown and described herein certain specific structure of the exemplary embodiments, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. A method of protecting cells that are susceptible to cell death as a result of physiological, environmental, traumatic or pathological causes in a mammalian subject comprising administering a cell penetrating peptide comprising at least 4 contiguous arginine residues to said subject to prevent, treat or ameliorate cell death.

2. A method of preventing, treating or ameliorating a condition selected from the group consisting inflammatory disorders, subarachnoid hemorrhage, macular degeneration, hearing loss, diabetic neuropathy, neuropathic pain, neurodegenerative diseases, ischemic injury after myocardial infarction, stroke, and reperfusion; and in autoimmune disorders comprising administering a cell penetrating peptide comprising at least 4 contiguous arginine residues to subject in need of such treatment.

3. The method of claim 1, wherein the cell penetrating peptide includes 4 to 30 contiguous arginine residues.

4. The method of claim 1, wherein the cell penetrating peptide includes 5 to 20 contiguous arginine residues.

5. The method of claim 1, wherein the cell penetrating peptide includes 6 to 12 contiguous arginine residues.

6. The method of claim 1 wherein the contiguous arginine residues are at the C-terminus.

7. The method of claim 1 wherein the contiguous arginine residues are at the N-terminus.

8. The method of claim 1 wherein the contiguous arginine residues are situated between the C-terminus and the N-terminus.

9. The method of claim 1 wherein at least 50% of the amino acid residues of the cell penetrating peptide are arginine residues.

10. The method of claim 1 wherein at least 70% of the amino acid residues of the cell penetrating peptide are arginine residues.

11. The method of claim 1 wherein at least 90% of the amino acid residues of the cell penetrating peptide are arginine residues.

12. The method of claim 1 wherein 100% of the amino acid residues of the cell penetrating peptide are arginine residues.

13. The method of claim 1 wherein the cell penetrating peptide comprises the amino acid sequence RRRRRRRRR (SEQ ID NO: 4), or is a derivative thereof.

14. The method of claim 1 wherein the cell penetrating peptide is a peptidomimetic.

15. A method of protecting cells that are susceptible to cell death as a result of physiological or pathological causes in a mammalian subject comprising administering a pharmaceutical composition comprising a cell penetrating peptide comprising at least 4 contiguous arginine residues and a pharmaceutically acceptable carrier to said subject to prevent, treat or ameliorate cell death.
